(19) United States
(12) Patent Application Publication
(10) Pub. No.: US 2018/0154094 A1
(43) Pub. Date: Jun. 7, 2018

(54) ARTIFICIAL VENTILATION APPARATUS ABLE TO DELIVER VENTILATION AND MONITORING WHICH ARE SPECIFIC TO THE PATIENTS RECEIVING CARDIAC MASSAGE

(71) Applicant: AIR LIQUIDE MEDICAL SYSTEMS, Antony, TX (US)

(72) Inventors: Eric JACQUOT, Antony (FR); Marceau RIGOLLOT, Montrouge (FR); Jean-Christophe RICHARD, Antony (FR)

(21) Appl. No.: 15/570,017

(22) PCT Filed: Feb. 26, 2016
(86) PCT No.: PCT/FR2016/050445

§ 371 Date: Oct. 27, 2017

(30) Foreign Application Priority Data
Apr. 28, 2015 (FR) 1553808

(51) Int. Cl. A61M 16/00

(52) U.S. Cl. CPC A61M 16/0048; A61M 16/10; A61M 16/0051; A61M 16/024

(57) ABSTRACT

The invention relates to a respiratory assistance apparatus (1) such as a medical ventilator, comprising a gas circuit (2, 16) with at least one inhalation branch (2) able to carry a respiratory gas intended to be administered to a patient under cardiac arrest during cardio pulmonary resuscitation; measurement means (6) suited to and designed for measuring at least one parameter indicative of said flow of gas and converting said at least one parameter indicative of said flow of gas into at least one signal indicative of said flow of gas; and a signal processing and control means (5, 8) suited to and designed for processing said at least one signal indicative of the flow of gas supplied by the measurement means (6) and deducing from said at least one signal indicative of the flow of gas information relating to the phases of compression and relaxation of a cardiac massage on the patient under cardiac arrest and controlling the motorised micro blower (40) and exhalation valve (19) accordingly in response to the phases detected.

### CROSS REFERENCE TO RELATED APPLICATION

This application is a 371 of International PCT Application PCT/FR2016/050445, filed Feb. 26, 2016, which claims priority to French Patent Application No. 1553808, filed Apr. 28, 2015, the entire contents of which are incorporated herein by reference.

### BACKGROUND

The invention relates to an artificial ventilation apparatus able to provide ventilatory assistance, in particular ventilation and monitoring, to help a first-aid worker or any other medical personnel, for example an emergency physician, a firefighter, a nurse or the like, when performing cardiac massage.

It is customary to use an artificial ventilation apparatus, also called a respiratory or ventilatory assistance apparatus, or more simply a medical ventilator, in order to provide respiratory assistance, that is to say artificial ventilation, to a person who is having difficulty breathing alone or is unable to breathe alone.

In particular, it is essential to ventilate a person during a cardiac arrest so as to continue supplying oxygen to the brain and to the rest of the body while the heart has stopped.

However, performing ventilation on a person in cardiac arrest while cardiac massage is being performed on this person is problematic, since this ventilation must not interrupt the chest compressions and/or must not be so great that it has adverse hemodynamic effects.

However, conventional ventilators are not designed for this situation, and they emit alarms and/or malfunction during the chest compressions.

Therefore, in practice, the medical personnel deliver insufflations via a ventilator or a bag valve mask (BVM), sometimes interrupting the chest compressions.

The continuity and regularity of the cardiac massage are factors of efficacy that determine the outlook for the patient in cardiac arrest. It is therefore never recommended to stop cardiac massage, even in order to provide the patient with insufflations of respiratory gas.

In addition, the insufflations delivered manually, via a BVM, or by a conventional ventilator are in most cases too aggressive, especially on account of the large volumes of gas that are administered, leading to a direct and recognized adverse effect on the efficacy of the chest compressions.

For this reason, apparatuses that avoid traditional insufflations, especially CPAP (Continuous Positive Airway Pressure) ventilators, have been proposed with a view to being used in cases of cardiac arrest. However, these apparatuses are not ideal since they are unable to ensure sufficient ventilation of the person in cardiac arrest, and the discontinuation of the compressions causes a discontinuation of all ventilation, which is undesirable.

Furthermore, most apparatuses capable of delivering mechanical ventilation during cardiac massage are unable to ensure this ventilation in an autonomous/automatic manner, whereas this complex clinical situation requires great ease of use. Indeed, these apparatuses sometimes require human intervention during each insufflation, which complicates the work of the medical personnel and sometimes leads to gas being administered at a poorly controlled pressure and/or volume.

If, during an insufflation of respiratory gas, the pressure and the volume generated are not correctly controlled, phenomena may arise that are damaging to the patient, for example undesirable gastric insufflation.

In other words, with the conventional apparatuses, it is not possible, in a reliable and simple manner, to deliver protective ventilation that does not exceed the recommended volume of gas.

In addition, during cardiac massage, the compressions of the thoracic cage generate a substantial but insufficient pulmonary ventilation, and the latter has to be supplemented by mechanical ventilation. When the chest compressions are interrupted, after spontaneous resumption of circulatory activity by the patient or after an electric shock for example, the ventilation decreases abruptly, whereas the oxygen requirements of the patient increase. Supplementary ventilation is then necessary, which entails complex manipulations for changing the ventilation mode in order to be able to remedy this lack of oxygen.

Thereafter, once this change of ventilation is effected and the patient is being suitably ventilated, a new cardiac arrest may occur, in this case, the chest compressions are resumed, and it is therefore once again necessary to manually modify the controls of the ventilation device in order to recover the ventilation mode that is the most suitable for cardiopulmonary resuscitation (CPR).

Given that a person may suffer several cardiac arrests in succession during his or her management by a medical team, the provision of optimal ventilation is complicated since it requires intervention by the medical team in order to adjust the apparatus each time said cardiac arrests occur. In emergency situations, however, the time spent on these adjustments is to the detriment of more important operations. Through lack of time, these adjustments may be forgotten about, ignored or neglected, which is unacceptable for obvious reasons of safety.

Generally speaking, most of the known mechanical ventilation apparatuses capable of being used during a cardiac massage do not have a specific mode adapted to this situation. They are simply equipped with inhalation triggers which often activate mistakenly and cause self-triggers and cycles that are damaging to the cardiac blood flowrate brought about by the chest compressions. Moreover, some of them do not afford the possibility of regulating a positive expiratory pressure (PEP), however indispensable this may be.

Finally, numerous acoustic and/or visual alarms with which these traditional apparatuses are equipped also mistakenly activate, for example alarms relating to pressure, volume or frequency, since they are developed for “traditional” applications, and the triggers and the alarms use the pressure signals and flowrate signals measured by the machine, which signals, for their part, are greatly disrupted by the cardiac massage.

The problem addressed is therefore to make available an artificial ventilation apparatus, that is to say a respiratory assistance apparatus, also called a medical ventilator, for solving all or some of the abovementioned problems and disadvantages, in particular a medical ventilator for detecting the performance of a cardiac massage on the patient who is in cardiac arrest, in particular the compression and decompression phases, with the aim of supplying the heart with the maximum mechanical energy generated by the chest compressions, by limiting the energy dissipated via the airways, including the lungs. The ventilator must also be capable of delivering a barometric ventilation at two pressure levels, on which the chest compressions can be superposed.

### SUMMARY

The solution of the invention thus concerns a respiratory assistance apparatus, also called a medical ventilator, comprising

a gas source or a gas supply device,

a gas circuit with at least one inhalation branch able to carry a respiratory gas intended to be administered to a patient in cardiac arrest during cardiopulmonary resuscitation,

measuring means able and designed to:
i) measure at least one parameter representative of said flow of gas,
ii) convert said at least one parameter representative of said flow of gas into at least one signal representative of said flow of gas,

and signal processing and control means able and designed to:
a) process said at least one signal representative of the flow of gas and supplied by the measuring means, and
b) deduce, from said at least one signal representative of the flow of gas, an item of information relating to the performance of a cardiac massage on the patient in who is in cardiac arrest, characterized in that
the gas source is a motorized micro-blower or the gas supply device comprises an inhalation valve, and
the signal processing and control means are configured to control the motorized micro-blower or the inhalation valve in such a way as to:
   i) respond to the detection of a compression phase by increasing the volume or the pressure of gas supplied by the motorized micro-blower or the inhalation valve, and
   ii) respond to the detection of a relaxation/decompression phase by decreasing the volume or the pressure of gas supplied by a motorized micro-blower or the inhalation valve.

Depending on the circumstances, the apparatus of the invention can comprise one or more of the following technical features:

the signal processing and control means are able and designed to transmit reference signals to the actuators, that is to say typically to the gas source, for example a micro-blower, or to one of the solenoid valves controlled by said signal processing and control means.

the signal processing and control means are able and designed to deduce, from said at least one signal representative of the flow of gas, at least one item of information relating to at least a compression phase and/or a relaxation/decompression phase of the thoracic cage during a cardiac massage performed on the patient.

the signal processing and control means are able and designed to deduce, from said at least one signal representative of the flow of gas, items of information relating to an alternation of compression phases and/or relaxation/decompression phases of the thoracic cage during a cardiac massage performed on the patient.

the signal processing and control means comprise an electronic card.

the gas source comprises a motorized micro-blower, also called a turbine or compressor, with an electric motor.

the gas source is a motorized micro-blower controlled by the signal processing and control means, said motorized micro-blower being in fluidic communication with the inhalation branch of the gas circuit.

alternatively, the gas supply device comprises an inhalation valve arranged on a gas supply conduit, said inhalation valve being controlled by the signal processing and control means. In this case, the gas source is external to the apparatus but is in fluidic communication with said gas supply conduit on which the controlled inhalation valve is arranged, for example a wall socket supplied with gas via a gas duct within a hospital building, and which is fluidically connected to the apparatus of the invention via a flexible conduit, for example.

it comprises a selection means by which a given ventilation mode specific to a cardiopulmonary resuscitation can be selected from among several stored ventilation modes. The selection of this mode makes it possible to start monitoring procedures and regulations specific to the ventilation during the cardiopulmonary resuscitation. The initiation of this mode also makes it possible to commence ventilation with predefined settings that have been registered beforehand by the user.

the measuring means are designed and able to measure at least one parameter representative of the flow of gas, chosen from among the gas pressure, the flowrate of gas insufflated to the patient, the flowrate of gas exhaled by the patient, and the speed of the micro-blower.

at least a part of the gas circuit, the signal processing means and the motorized micro-blower or the inhalation valve of the supply device are situated in a rigid shell, that is to say an outer envelope forming the covering or cowling of the apparatus.

the gas circuit comprises an internal portion arranged in the rigid shell and an external portion situated outside the rigid shell and forming all or part of the inhalation branch.

it additionally comprises a man-machine interface able to display items of information including at least one item of information relating to the performance of a cardiac massage on the patient who is in cardiac arrest.

the gas circuit additionally comprises an exhalation branch in fluidic communication with the atmosphere via a gas outlet orifice and having an exhalation valve and an exhalation flowrate sensor.

the exhalation flowrate sensor is preferably a hot-wire sensor.

the external portion of the inhalation branch of the gas circuit is in fluidic communication with a respiratory interface, in particular a breathing mask or an intubation cannula.

the exhalation flowrate sensor is arranged between the exhalation valve and the gas outlet orifice communicating with the atmosphere.

alternatively, the exhalation flowrate sensor is arranged between the exhalation valve and the respiratory interface supplying the patient with gas.

the signal processing and control means are configured to control the motorized micro-blower or the inhalation valve and the exhalation valve as a function of signals received from the measuring means and from the exhalation flowrate sensor.

the signal processing and control means are configured, that is to say designed and able, to control the motorized micro-blower or the inhalation valve of the gas source in such a way as to increase the volume or the pressure of gas supplied by the motorized micro-blower or the inhalation valve in response to the detection of a compression phase, with the aim of amplifying the pressure peak caused by the chest compression of the patient, by slowing down the ejection of a volume of air contained in the respiratory system of said patient.

the signal processing and control means are configured, that is to say designed and able, to control the motorized micro-blower or the inhalation valve of the gas source in such a way as to decrease the volume or the pressure of gas supplied by the motorized micro-blower or the inhalation valve in response to the detection of a relaxation/decompression phase, in such a way as to favor a passive return of the thoracic cage of the patient to the equilibrium position.

the signal processing and control means are configured to control the exhalation valve in such a way as to limit or stop the flowrate of gas passing through the exhalation valve in response to the detection of a compression phase or a relaxation/decompression phase.

the signal processing and control means comprise at least one microprocessor using at least one algorithm.

it additionally comprises data storage means, that is to say memory means.

the data storage means comprise one or more memories, in particular a flash memory or the like.

the data storage means are configured to store several ventilation modes including at least one given ventilation mode specific to a cardiopulmonary resuscitation.

the measuring means are designed and able to measure at least one parameter representative of the flow of gas, chosen from among the gas pressure, the flowrate of gas insufflated to the patient, the flowrate of gas exhaled by the patient, and the speed of the micro-blower.

the internal portion of the gas circuit is in fluidic communication with a gas source, in particular a micro-blower, so as to be supplied with gas delivered by said gas source.

the measuring means are arranged on the gas circuit situated inside or outside the rigid shell, so as to perform the desired measurements there.

it comprises several measuring means, some of which are arranged on the internal portion of the gas circuit, while others are arranged on the external portion of the gas circuit.

the measuring means comprise one or more sensors.

the measuring means comprise a sensor arranged to perform measurements in the gas circuit, preferably in the inhalation branch of the gas circuit.

It additionally comprises a man-machine interface which is able, that is to say designed, to display items of information including at least one item of information relating to the performance of a cardiac massage on the patient who is in cardiac arrest.

the man-machine interface comprises a display screen.

the inhalation branch of the gas circuit is in fluidic communication with a respiratory interface, in particular a breathing mask or an intubation cannula.

the inhalation branch of the gas circuit comprises a flexible tube or conduit.

the gas source is a source of air (ca. 21% by volume of O₂) or of oxygen-enriched air (>21% by volume of O₂).

the signal processing means comprise at least one microprocessor, preferably arranged on an electronic board.

the signal processing means comprise at least one microcontroller, preferably at least one microcontroller using at least one algorithm.

the shell comprises at least one carrying handle to facilitate the transport of the apparatus by a user.

the shell comprises at least one securing device allowing the ventilation apparatus to be secured on a support, for example a bar inside an emergency vehicle, or a rung of a bed or stretcher.

it comprises means for supply of electric current, for example one or more batteries or similar, or one or more cables and one or more connections to the mains supply.

it additionally has regulation and selection means, for example a push button, an activation key, a slide or similar, allowing the medical personnel to act on the ventilator, for example in order to inform the ventilator of the performance of a cardiac massage, to confirm, for the ventilator, a detection of a cardiac massage, to inform the ventilator of the type of respiratory interface used (mask, intubation tube, etc.), to modify one or more mechanical ventilation parameters that are proposed automatically by the ventilator, or for other purposes.

Generally speaking, as regards the ventilation mode specific to cardiopulmonary resuscitation, this can be a volumetric or barometric mode, preferably associated with a minimal pressure of ventilation, for example 5 cm H₂O.

Advantageously, it is a barometric mode that ensures alternating regulation at two pressure levels, comprising a low pressure level (PB) and a high pressure level (PH), with PH>PB, for example a low pressure of the order of 5 cm H₂O, and a high pressure of the order of 15 cm H₂O.

The ventilation mode specific to cardiopulmonary resuscitation is able to ensure ventilation of a patient from the start to the end of the intervention in an environment requiring little or no human intervention during the various phases.

In addition to this ventilation mode specific to cardiopulmonary resuscitation, the respiratory assistance apparatus of the invention has other modes of conventional ventilation, for example one or more modes of volumetric ventilation (VAC), barometric ventilation (VPC, VSAI, CPAP, Duo-Levels, etc.) and/or intermittent ventilation (VACI, PVACI).

To improve the blood circulation on the basis of the ventilation, the apparatus of the invention is configured to modulate the opening of the exhalation valve and the acceleration of the micro-blower, during the time of the chest compression, in such a way as to limit the dissipation of energy by slowing down the ejection of the volume of air contained in the respiratory system, in order to amplify the pressure transmitted to the heart.

During the decompression time or the rise of the thoracic cage, the apparatus of the invention is configured to modulate the speed of the micro-blower in such a way as to favor a passive return of the thoracic cage to the equilibrium position, such that the intrathoracic pressure remains negative, thus permitting venous return and filling of the heart.

The succession of the compression and decompression phases, for example at a frequency of 100 times per minute, with alternation between positive pressure and negative pressure, makes it possible to generate cardiac flowrate by a pump effect. In other words, by modulating the speed of the micro-blower to the rhythm of the chest compressions and the opening of the exhalation valve, the apparatus according to the invention acts as an amplifier of the pressure variations generated by the chest compressions, as opposed to an apparatus seeking to regulate a constant pressure at one or two pressure levels, for example CPAP or PAC.

In practice, the apparatus provides modulation, to the frequency of the cardiac massage, of the speed of the micro-blower and the opening of the exhalation valve, by contrast to a conventional pressure mode.

Indeed, with CPAP or a conventional pressure mode, the principle is to keep the pressure as constant as possible, irrespective of the disturbances, in particular of the chest compressions of the CPR. However, this conventional approach does not permit optimization of the pressures generated by the chest compressions, which are key to the efficacy of the CPR.

Finally, in order to supplement the ventilation which is insufficient in the case of chest compressions alone, the apparatus of the invention is configured so as to make it possible to improve/increase the ventilation delivered by the ventilator by the addition of pressure cycles (as in controlled pressure) at the chosen frequency. The chest compressions on these cycles remain possible, which distinguishes the apparatus of the invention from a conventional apparatus functioning according to a pressure or volume mode.

It will be noted that, within the scope of the present invention, the term “means” is regarded as being strictly equivalent to the term “device”. Hence, “measuring means” is equivalent to “measuring device”; “display means” is equivalent to “display device”; “processing means” is equivalent to “processing device”; “data storage means” is equivalent to “data storage device”, etc.

### BRIEF DESCRIPTION OF THE DRAWINGS

The present invention will now be described in more detail with reference to the attached figures, in which:

FIG. 1 shows a first embodiment of a respiratory assistance apparatus according to the present invention, in which the gas source is a micro-blower,

FIG. 2 shows a second embodiment of a respiratory assistance apparatus according to the present invention, in which the gas source is a supply device comprising a controlled valve, and

FIG. 3 is a graph showing the intrathoracic pressure obtained using a respiratory assistance apparatus according to the present invention.

### DESCRIPTION OF PREFERRED EMBODIMENTS

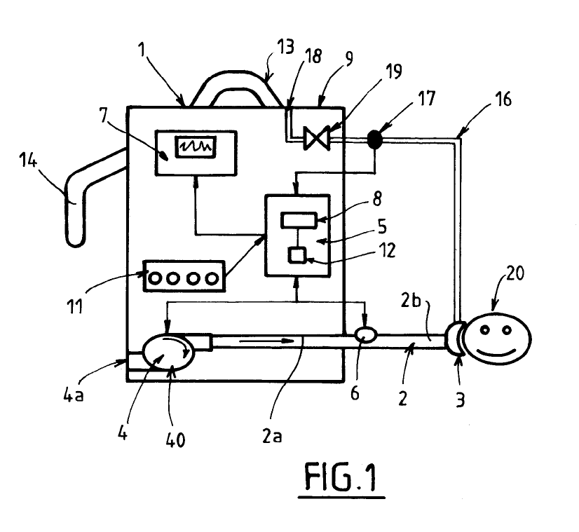

FIG. 1 is a schematic view of a first embodiment of a ventilatory assistance apparatus or medical ventilator 1 according to the present invention.

The ventilator 1 comprises a gas source 4, which is here a motorized micro-blower 40, also called a turbine, delivering a flow of respiratory assistance gas, typically a flow of air or of oxygen-enriched air, in the inhalation branch 2 of a ventilatory circuit 2, 16, also called the patient circuit, comprising one or more passages, conduits or gas lines fluidically connecting the ventilator 1 to the airways of a patient 20, by way of a patient interface 3, such as a breathing mask or an intubation cannula or probe.

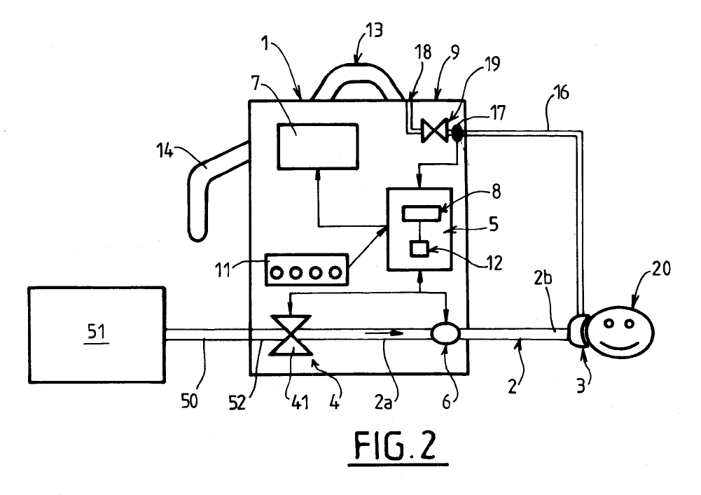

Alternatively, as is illustrated in the second embodiment of FIG. 2, the gas source 4 can comprise an inhalation valve 41 supplied with gas from a gas duct 50, which is itself supplied from a gas reservoir 51, for example a hospital network of gas ducts, a cylinder or an external gas compressor. The gas duct 50 is in fluidic communication with an internal gas conduit 52 which is inside the ventilator 1 and on which the inhalation valve 41 is arranged.

The respiratory gas is typically air or oxygen at a pressure of several bar absolute, typically of the order of approximately 4bar absolute. This inhalation valve is controlled by the signal processing and control means 5, 8 of the apparatus 1.

Measuring means 6 are provided, such as one or more sensors, which are able and designed to measure at least one parameter representative of the flow of gas, chosen from among the gas pressure, the flowrate of gas insufflated by the respirator, the flowrate of gas exhaled by the patient 20, and the speed of rotation of the micro-blower 40, and to deliver at least one signal representative of said at least one measured parameter.

For example, the parameter representative of the flow of gas is the pressure of the gas in the ventilatory gas circuit 2, and the measuring means 6 comprise a pressure sensor whose pressure tapping is arranged in said ventilatory circuit 2 in such a way as to permit measurement of the gas pressure prevailing in all or part of the ventilatory circuit 2.

In the first embodiment of FIG. 1, the pressure tapping serving as measuring means 6 is arranged outside the shell 9 of the ventilator 1. However, it can also be situated within the ventilator 1. Moreover, in the embodiment of FIG. 2, the pressure tapping serving as measuring means 6 has been shown within the shell 9 of the ventilator 1.

Once the one or more parameters representative of the flow of gas have been measured, the corresponding one or more signals are transmitted to and analyzed by signal processing and control means 5, 8 which can then deduce:

that a cardiac massage is in the process of being performed on the patient 20 and in particular can determine if the phase in progress is a compression phase or a relaxation phase of the thoracic cage;

the volume of gas insufflated by the ventilator 1 to the patient 20, in the course of the mechanical ventilation cycles and during the phases of relaxation of the thoracic cage. The volumes of insufflated gas can be added together over a given period of time, for example 1 minute. Of course, the addition can be performed for longer than 1 minute or, conversely, for less than 1 minute.

the volume of gas exhaled by the patient 20, in the course of the mechanical ventilation cycles and during the phases of relaxation of the thoracic cage. Here too, the volumes of exhaled gas can be added together over a given period of time, for example 1 minute. Of course, the addition can be performed for longer than 1 minute or, conversely, for less than 1 minute.

The one or more signals representative of the flow of gas are transmitted by the measuring means 6 to the signal processing and control means 5, 8 via a suitable link, that is to say electrical links such as cables or the like.

Moreover, the signal processing and control means 5, 8 are able and designed to:

i) process the signal corresponding to the parameter representative of the flow of gas and, for example, detect one or more positive or negative variations greater than one or more threshold values representative of the phases of compression or relaxation of the thoracic cage in the course of a cardiac massage. These threshold values are recorded in a storage memory 12, for example a flash memory. These threshold values can be numerical values, tables of values, curves, etc.

ii) integrate, on the signal corresponding to the parameter representative of the flow of gas, the gas flowrate generated by the ventilator 1 during the chest compressions and the cycles generated by the machine.

iii) integrate, with respect to time, the signal corresponding to the parameter representative of the flow of gas, and the gas flowrate generated by the ventilator 1 during the chest compressions and the cycles generated by the ventilator 1.

iv) integrate, with respect to time, the signal corresponding to the parameter representative of the flow of gas, and the gas flowrate exhaled by the patient 20 during the chest compressions and the cycles generated by the ventilator 1.

To do this, the signal processing means 5, 8 preferably comprise a microprocessor 8 programmed in particular with one or more processing algorithms, as is explained in detail below.

The ventilator 1 and its components, requiring power in order to function, are supplied directly or indirectly with electrical current from one or more rechargeable or non-rechargeable batteries, from the electricity supply of the emergency vehicle that it equips or from the mains supply, hence at a voltage that can be as much as about 230 V. If necessary, it can incorporate a current converter designed to reduce the supply voltage to a use voltage that is of a lower value.

In addition, a man-machine interface 7, such as a display screen, makes it possible to display, and thus allows the user to view, the items of information calculated by the signal processing and control means 5, 9.

Also provided are regulation or selection means 11, for example push buttons or rotary knobs, slides, activation or selection keys or similar, allowing the medical personnel to inform the ventilator 1 of the performance of a cardiac massage and/or to confirm, for the ventilator 1, the detection made of the performance of a cardiac massage, and to inform the ventilator of the type of interface with the patient, for example mask, intubation tube, etc.

Thus, at least one regulation or selection means 11 is provided which can be activated by the operator in order to be able to select a given ventilation mode, specific to a cardiopulmonary resuscitation, from among several stored ventilation modes.

These regulation or selection means 11 also make it possible, if need be, to modify the mechanical ventilation parameters that are proposed automatically by the ventilator 1, or, depending on the embodiment in question, to be able to inform the ventilator 1 of a change in the nature of the gas used, for example the move from air to an air/oxygen mixture, or a change in the oxygen content of an air/oxygen mixture.

As can be seen in FIGS. 1 and 2, at least a part of the gas circuit 2, the signal processing means 8 and the gas source 4 are arranged in a cowling or a rigid shell 9 which forms the outer envelope of the apparatus 1. This shell 9 includes or moreover supports other components such as the man-machine interface 7, the one or more memories 12, the regulation and selection means 11, etc.

The gas circuit 2, 16 comprises either an inhalation branch 2 on its own or an inhalation branch 2 and an exhalation branch 16 which, for example, are connected to each other by a Y-shaped piece.

The inhalation branch 2 in fact comprises two distinct portions, namely an internal portion 2a arranged in the rigid shell 9, for example a gas conduit, and an external portion 2b situated outside the rigid shell 9 and including, for example, a flexible hose.

The internal portion 2a of the gas circuit 2 is in fluidic communication with the gas source 4, typically a motorized micro-blower having an air intake or inlet 4a communicating with the ambient atmosphere, in such a way as to supply said internal portion 2a with air, optionally enriched in oxygen.

The motorized micro-blower 4 is controlled by control means 5, for example an electronic board with microprocessor, such as a microcontroller, using one or more algorithms.

The signal processing and control means 5, 8 are in fact configured to control the motorized micro-blower 4 as a function of the signals transmitted by the signal processing means 5, 8, in such a way as to permit in particular a synchronization between the delivery of gas to the patient 20 and the cardiac massages.

In other words, the signal processing and control means 5, 8 are configured to control the exhalation valve 19 as a function of the signals transmitted by the signal processing means 5, 8, in such a way as to permit in particular a synchronization between the delivery of gas to the patient 20 and the cardiac massages.

Moreover, the external portion 2b of the gas circuit 2 situated outside the rigid shell 9 is for its part in fluidic communication, at the upstream end, with the internal portion 2a of the gas circuit 2 and, at the downstream end, with the respiratory interface 3, so as to ensure fluidic continuity between the gas source 4 and the patient 20 and to allow the respiratory gas, e.g. the air arriving from the turbine, to reach the airways of said patient.

Here, the measuring means 6, typically one or more sensors, are arranged on the external portion 2b of the inhalation branch 2 of the gas circuit 2, 16 situated outside the rigid shell 9, in order to perform the desired measurements, for example of pressure and/or flowrate, within said external portion 2b. In this case, the link between the measuring means 6 and the processing means 5, and hence the transfer of the measuring signals, is effected by wired connections, for example.

Optionally, the shell 9 can also comprise at least one carrying handle 13 to facilitate the transport of the apparatus 1 by the user, as is essential in some emergency situations, and/or a securing device 14 allowing the ventilation apparatus 1 to be secured on a support, for example a bar inside an emergency vehicle, or a rung of a bed or stretcher.

Moreover, the exhalation branch 16 comprises an exhalation flowrate sensor 17, for example a hot-wire sensor, connected electrically to the signal processing and control means 5, 8, and also an exhalation valve 17 controlled by the signal processing and control means 5, 8. At its downstream end, the exhalation branch 16 communicates with the atmosphere via a gas outlet orifice 18, while its upstream end is connected to the inhalation branch 2, via a Y-shaped piece, or directly to the patient interface 3.

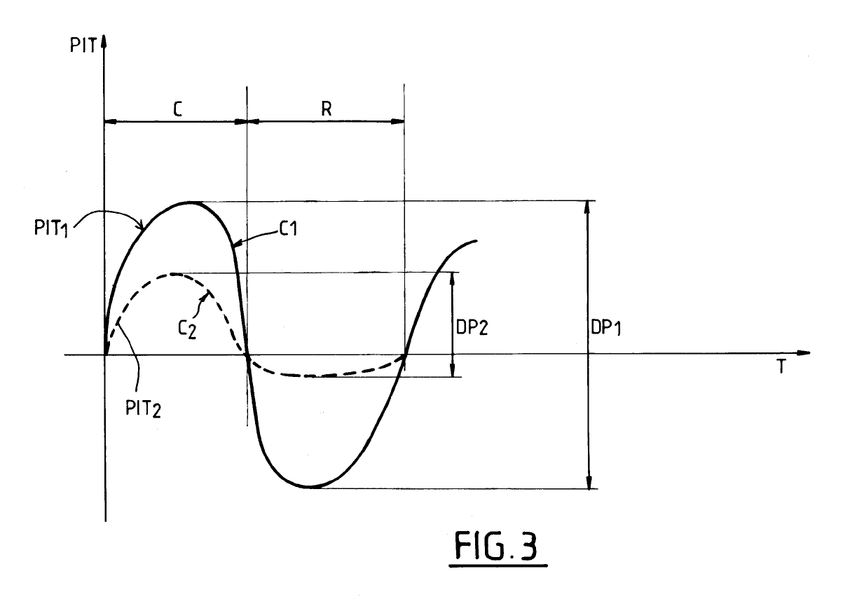

FIG. 3 is a graph illustrating the intrathoracic pressure obtained using the respiratory assistance apparatus according to the present invention.

A cardiac massage can be considered as an alternation and succession of compression phase C and relaxation phase R (i.e. decompression), during which the intrathoracic pressure (PIT) varies over the time (T) between maximum and minimum values, as is illustrated by the curves in FIG. 3.

More precisely, the intrathoracic pressure PIT1 obtained using a respiratory assistance apparatus according to the present invention permits a pressure variation DPI (curve C1) greater than the pressure variation DP2 calculated on the basis of the intrathoracic pressure PIT2 (curve C2) generated by a conventional ventilation apparatus.

During the thoracic cage compression phase C, which is determined by the signal processing and control means 5, 8, the ventilator controls, by virtue of these same signal processing and control means 5 and 8, the motorized micro-blower 40 or the inhalation valve 41 and the exhalation valve 19 of FIGS. 1 and 2, in such a way as to slow down the ejection of the air volume contained in the respiratory system and thus maximize the intrathoracic pressure 3 during this compression phase C.

Moreover, during the thoracic cage decompression/relaxation phase R, which is determined by the signal processing and control means 5, 8, the ventilator controls, by virtue of these same signal processing and control means 5 and 8, the motorized micro-blower 40 or the inhalation valve 41 and the exhalation valve 19 of FIGS. 1 and 2, in such a way as to limit the delivery of gas to the respiratory system and thus favor a passive return of the thoracic cage to the equilibrium position and cause a negative intrathoracic pressure 3 during this decompression/relaxation phase R.

During the cardiac massage, the apparatus of the present invention thus delivers a barometric ventilation between two pressure levels, namely a low pressure level (PB) and a high pressure level (PH), with PB<PH. The cardiac massage continues both during the application of the low pressure and during the application of the high pressure. The specific control of the actuators, i.e. motorized micro-blower 40 or inhalation valve 41 and exhalation valve 19 in particular, during the phases of compression C and relaxation R of the thoracic cage is effected both at the low pressure level PB and also at the high pressure level PH.

The barometric mode thus ensures a regulation of alternating pressure between the two pressure levels PH and PB, for example with a low pressure PB of the order of 5 cm H₂O, and a high pressure PH of the order of 15 cm H₂O.

This ventilation mode specific to the cardiopulmonary resuscitation makes it possible to ventilate a patient in cardiac arrest right from the start of the intervention, and in an environment requiring little or no human intervention during the different phases.

Generally, according to the present invention, the signal processing and control means 5, 8 are configured, that is to say designed and able, to control the motorized micro-blower 40 or, depending on the embodiment in question, the inhalation valve 41 in such a way as to adjust the volume or the pressure of gas supplied by the motorized micro-blower 40 or the inhalation valve 41 depending on the detected phase of cardiac massage during a cardiopulmonary resuscitation (CPR).

Thus, in response to the detection of a compression phase, the signal processing and control means 5, 8 of the apparatus of the invention will drive the motorized micro-blower 40 or the inhalation valve 41 in such a way as to obtain an increase in the volume or the pressure of gas supplied by the motorized micro-blower 40 or the inhalation valve 41, whereas, in response to the detection of a relaxation/decompression phase, said signal processing and control means 5, 8 will drive the motorized micro-blower 40 or the inhalation valve 41 in such a way as to decrease the volume or the pressure of gas supplied by the motorized micro-blower 40 or the inhalation valve 41.

By detection of the thoracic compression and decompression phases effected during the performance of cardiac massage on a patient in cardiac arrest, such control will then make it possible to supply the heart with a maximum mechanical energy generated by the thoracic compressions, limiting the energy dissipated via the airways, including the lungs, in particular by virtue of careful adjustment of the volume and/or the pressure of gas supplied by the motorized micro-blower 40 or the inhalation valve 41 depending on the detected phase of cardiac massage.

The respiratory assistance apparatus according to the present invention can be used in the context of ventilation of a person who is in cardiac arrest and who is receiving cardiac massage.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A respiratory assistance apparatus (1) comprising:
a gas source (4) or a gas supply device (41, 55),
a gas circuit (2, 16) with at least one inhalation branch (2) able to carry a respiratory gas intended to be administered to a patient in cardiac arrest during cardiopulmonary resuscitation,
measuring system (6) able and designed to:
i) measure at least one parameter representative of said flow of gas,
ii) convert said at least one parameter representative of said flow of gas into at least one signal representative of said flow of gas,
and a signal processing and control system (5, 8) able and designed to:
a) process said at least one signal representative of the flow of gas and supplied by the measuring system (6), and
b) deduce, from said at least one signal representative of the flow of gas, an item of information relating to the performance of a cardiac massage on the patient who is in cardiac arrest, wherein
the gas source (4) is a motorized micro-blower (40) or the gas supply device (41, 55) comprises an inhalation valve (41), and
the signal processing and control system (5, 8) is configured to control the motorized micro-blower (40) or the inhalation valve (41) in such a way as to:
i) respond to the detection of a compression phase by increasing the volume or the pressure of gas supplied by the motorized micro-blower (40) or the inhalation valve (41), and
ii) respond to the detection of a relaxation/decompression phase by decreasing the volume or the pressure of gas supplied by a motorized micro-blower (40) or the inhalation valve (41).

2. The apparatus of claim 1, wherein the signal processing and control system (5, 8) comprises an electronic board and is able and designed to deduce, from said at least one signal representative of the flow of gas, at least one item of information relating to at least a compression phase and/or a relaxation/decompression phase of a thoracic cage during a cardiac massage performed on the patient.

3. The apparatus of claim 1, wherein the gas source (4) is a motorized micro-blower controlled by the signal processing and control system, said motorized micro-blower being in fluidic communication with the at least one inhalation branch (2) of the gas circuit (2, 16).

4. The apparatus of claim 1, wherein having the gas supply device (41, 55), the gas supply device comprising an inhalation valve (41) arranged on an internal gas conduit (55), said inhalation valve being controlled by the signal processing and control system (5, 8).

5. The apparatus of claim 1, further comprising at least one selection system (11), which can be activated by the operator, designed to select a given ventilation mode specific to one cardiopulmonary resuscitation from among several stored ventilation modes.

6. The apparatus of claim 1, wherein the measuring system (6) is designed and able to measure at least one parameter representative of the flow of gas, said at least one parameter representative of the flow of gas being chosen from among a gas pressure, a flowrate of gas insufflated to the patient, a flowrate of gas exhaled by the patient, and a speed of the micro-blower (4).

7. The apparatus of claim 1, wherein at least a part of the gas circuit (2, 16), the signal processing system (5, 8), and either the motorized micro-blower (4) or the inhalation valve (41) of the gas supply device (41, 55), are situated in a rigid shell (9).

8. The apparatus of claim 7, wherein the gas circuit (2, 16) comprises an internal portion (2a) arranged in the rigid shell (9) and an external portion (2b) situated outside the rigid shell (9) and forming all or part of an inhalation branch (2).

9. The apparatus of claim 1, further comprising a man-machine interface (7) able to display items of information including at least one item of information relating to a performance of a cardiac massage on the patient who is in cardiac arrest.

10. The apparatus of claim 1, wherein the gas circuit (2, 16) additionally comprises an exhalation branch (16) in fluidic communication with the atmosphere via a gas outlet orifice (18) and having an exhalation valve (19) and an exhalation flowrate sensor (17).

11. The apparatus of claim 8, wherein the external portion (2b) of the inhalation branch (2) of the gas circuit (2, 16) is in fluidic communication with a respiratory interface (3), in particular a breathing mask or an intubation cannula.

12. The apparatus of claim 10, wherein the signal processing and control system (5, 8) is configured to control the motorized micro-blower (4) or the inhalation valve and the exhalation valve (19) as a function of signals received from the measuring system (16) and from the exhalation flowrate sensor (17).

13. The apparatus of claim 10, wherein the signal processing and control system (5, 8) is configured to control the exhalation valve (19) in such a way as to limit or stop a flowrate of gas passing through the exhalation valve (19) in response to a detection of a compression phase or a relaxation/decompression phase.

14. The apparatus of claim 1, wherein the signal processing and control system (5, 8) comprises at least one microprocessor using at least one algorithm.

15. The apparatus of claim 1, further comprising a data storage configured to store several ventilation modes including at least one given ventilation mode specific to a cardiopulmonary resuscitation.
